(19) United States
(12) Patent Application Publication
(10) Pub. No.: US 2014/0342019 A1
(43) Pub. Date: Nov. 20, 2014

(54) Preparation of Personal Care Substances

(71) Applicants: Tiffany Andersen, Las Vegas, NV (US); Robert Allen Barlett, Tulalip, WA (US)

(72) Inventors: Tiffany Andersen, Las Vegas, NV (US); Robert Allen Barlett, Tulalip, WA (US)

(21) Appl. No.: 14/276,709

(22) Filed: May 13, 2014

Related U.S. Application Data
(60) Provisional application No. 61/823,227, filed on May 14, 2013.

(51) Int. Cl. A61K 33/24; A61Q 19/00; A61K 8/19

(52) U.S. Cl. CPC A61K 33/24; A61K 8/19; A61Q 19/00; A61K 2800/805
USPC 424/649; 514/769

(57) ABSTRACT

Methods preparing a personal care substance in fluid form from gemstones or gold. The gemstones or gold are comminuted and dissolved. Solvent is removed by distillation, leaving an oily residue usable in the personal care substance. The personal care substance may include fulvates and humates, or dissolved gold. The personal care substance may comprise an ozonated oil.

### REFERENCE TO RELATED APPLICATION

This application claims the benefit of U.S. Provisional Application No. 61/823,227, filed on May 14, 2013.

### FIELD OF THE INVENTION

The present invention relates to personal care substances for topical application to the skin and for ingestion.

### BACKGROUND OF THE INVENTION

Personal care substances have long been sought by consumers, and have found an enthusiastic market. These personal care substances may be applied externally to the skin, or in some case, ingested orally for internal administration. While many personal care substances have been developed relatively recently, others which rely on historically known ingredients may also be developed.

### SUMMARY OF THE INVENTION

The present invention provides personal care substances which utilize certain specific ingredients which have traditionally or historically been utilized. Accordingly, the invention contemplates processes for preparing personal care substances, based on mineral based ingredients such as those in gemstones and gold, and organically based ingredients such as organic solvents, eggs, and others. The personal care substances are in fluid form, including a carrier fluid together with active ingredients. The final fluids may be topically applied, or may be used as additives to other personal care substances.

### DETAILED DESCRIPTION

Referring first to fluid preparations, according to at least one aspect of the invention, there is shown a process for preparing fluids, which will variously be referred to as “oils” and “elixirs”, from garnet, diamond, ruby sapphire, and other gems. The process utilizes a solvent, which may be a distillate of a metallic acetate composition. The solvent acted upon in this process is prepared by the destructive distillation of a metal acetate salt. The preferred salt is lead acetate but the acetates of zinc and sodium may also be used.

The solvent is prepared by the dry distillation of one of the selected metal acetates gradually at a maximum temperature of 700 degrees Celsius. Special attention is paid to the cooling system used to condense the vapor produced in the distillation. The collected distillate is placed into a fractional distillation train and the fraction distilling over up to 56 degrees Celsius is collected. The resulting clear, volatile liquid is preserved in a sealed glass container stored in a cool place.

Oils and tinctures may be prepared in the following way. A quantity of gems sufficient for an extraction is crushed and calcined. Some of the previously prepared solvent is added after cooling the crushed and calcined gem material to about room temperature. The mixture is circulated in a suitable vessel such as a pelican. For the purposes of this application, a pelican is a circulatory distillation vessel with two side-arms feeding condensed vapors back into the body. The pelican is so-called as it resembles a pelican pecking at its breast to feed its hatchlings. When a tincture forms, the tincture is removed, and the solvent is removed from the tincture by distillation. A thicker fluid, appearing as an oil, is left behind. This oil may be added to a quantity of 96% alcohol to form a tincture. The resulting substance, which may be incorporated into a personal care substance, is thus made available as the tincture.

Alternatively, an elixir may be prepared in the following way. The thicker fluid from the prior process is calcined. Ashes are extracted using filtered rainwater. The mixture is filtered and allowed to form crystals. The mixture, with crystals, is blended with a quantity of the priorly described tincture, and circulated in the pelican. Alcohol is then removed by distillation. Alcohol recovered after the distillation is added to the solid residue, and distillation is repeated. After a number of iterations, crystals will form on the retort head. The crystals are collected and combined with alcohol previously removed by distillation. The mixture is maintained at 110 degrees F. for several months. After this period, the liquid is filtered, and with solids thus removed, is available as an elixir.

A mineral fulvate complex from ocean water may be prepared as follows. The following process is appropriate for about one gallon of ocean water. In a separate beaker containing pure sodium hydroxide crystals, water is added until a pH of 11 is rached. This solution is added in individual drops to the ocean water. The mixture is constantly stirred until a pH of 10.5 is reached. The mixture is allowed to settle for about four hours. Supernatant is removed. The precipitate is washed two or three times. After the supernatant is removed, fulvic or humic acid is added in individual drops to further lower the pH to a suitable range for mineral humates or fulvates to form.

Plant based fulvate and humate complexes may be prepared from aloe and other plants. A preparation of mineral precipitate from aloe is described, using one gallon of fluids taken from the interior of aloe leaves. Sodium hydroxide is added until a pH of 11 is reached. The aloe mixture is boiled for four hours under medium heat. Additional sodium hydroxide in water is provided to replace that which boils off. After allowing the aloe mixture to cool, residue is filtered and placed into a separate beaker. Either fulvic acid or humic acid or a combination is added until the pH is lowered to a desired result. Supernatant is filtered out. The remaining plant based fulvate and/or humate complex may be used as desired.

A fluid which for various reasons is referred to as oil of egg, including fulvates and humates from plants, may be prepared as follows.

This process describes the preparation of an oil derived from eggyolks using supercritical fluid extraction. One hundred twenty eggs are dehydrated to obtain powdered yolk. This is done by hardboiling the eggs, recovering the yolks, dehydrating for several hours until all water is removed, and comminuting them into a powder. The dehydrated egg yolk powder is placed into a glass tube. A supercritical fluid, such as carbon dioxide or butane, is injected into the top of the glass tube. An oil or fluid will evacuate from the bottom and is collected in a collection dish. The oil can be further refined until all solids are removed.

Spagyric mineral salts may be combined with certain metal acetates to generate a useful additive. Distilled metal acetate is used to extract a fluid bearing comminuted crystals. A spagyric method is one in which essential components of a material are first separated then purified by appropriate techniques and finally reunited into a new homogenous whole. The resulting product is held to have enhanced effectiveness compared to the crude or merely chemically purified substance. As an example of the process, a preparation of minerals called Schussler Cell Salts will be described. The Schussler Cell Salts include potassium phosphate, sodium sulfate, potassium chloride, calcium fluoride, magnesium phosphate, potassium sulfate, sodium phosphate, calcium sulfate, silicon dioxide, calcium phosphate, sodium chloride, and iron phosphate. These salts are required in USP of NF grade. In addition the metal acetate of the parent metal of the particular salt is also required. For example there are three salts of calcium listed above, therefore calcium acetate is required to produce all three. In all, five acetates are required to produce the twelve salts, including potassium acetate, sodium acetate, magnesium acetate, calcium acetate, and iron acetate. It is preferred that the source of the metal acetates is of natural origin such as limestone for calcium or natron for sodium, the respective acetate being produced by known methods.

An exemplary process begins with dry distillation of one of the selected metal acetates, with gradual heating up to a maximum temperature of 700 degrees Celsius. Special attention is paid to the cooling system used to condense the vapor produced in the distillation. The collected distillate is placed into a fractional distillation train and the fraction distilling over up to 56 degrees Celsius is collected. The resulting clear, volatile liquid is preserved in a sealed glass container, preferably labeled “Volatile”, and is stored in a cool place.

The remaining dark liquid in the distillation flask is extracted with ethyl ether or petroleum ether. The ether layer is filtered through a 40 micron glass filter, then gently evaporated to a fluid or oil. This oil is collected and preserved in a glass container preferably labeled as an oil of the applicable parent metal.

The fraction labeled “Volatile” is purified by redistillation. The oil is purified by dissolving in dried ethanol allowing it to stand for several days, then is filtered and evaporated to an oil. At this point, the essential components of the mineral are separated and purified in the form of a volatile liquid, an aromatic oil, and the USP grade mineral salt.

The oil and the volatile liquid are combined in a glass container. in an example here, the three calcium salts are prepared. Calcium acetate has been distilled and the purified volatile and oil recombined. Each of the three USP grade calcium salts (calcium fluoride, calcium sulfate and calcium phosphate) are finely ground then placed into separate glass vessels and just saturated with the combined volatile and oil solution, sealed and allowed to stand in a warm place (40 degrees Celsius maximum) Two weeks of such digestion is a minimum (longer is preferred) before use. If the digesting salt appears to have dried, more of the liquid is added until just saturated. The final product will be homogenous and waxy in appearance, similar to butter.

This final product is now the starting point for homeopathic dilutions. The preferred diluent is 50% ethanol up to three times and 10 to 15% ethanol for all higher dilutions.

The following process describes the preparation of an oily product through the extraction of a pure gold precipitate with a specially prepared solvent mainly consisting of ethyl alcohol to which a sublimated salt has been added. For various reasons, the resulting product will be called “the oil of gold”, and represents a class of materials of medical interest called “potable gold”.

The process utilizes gold, with a preferred source being gold ore that has not been melted (e.g., placer mined nuggets or flakes). Pure gold which has been refined by fire can also be used. Gold of lesser karat value than 24 must be refined, with refinement through the use of antimony preferred.

A solvent used in this process is prepared by sublimating sal ammoniac (NH₄Cl) several times until a yellow or orange crystal forms. The preferred salt is sal ammoniac but certain other salts may be substituted. Pure gold is dissolved into aqua regia and the resulting solution is filtered through a 40 micron glass frit. The filtered liquid is slowly evaporated on a low heat until it crystallizes. The crystals are then re-crystallized from a 1M solution of hydrochloric acid and then re-crystallized from distilled water. The resulting deep golden orange crystals of hydrogen tetrachloroaurate are then gently surface dried and stored in an airtight glass container kept cool and out of direct light. Care must be taken as the crystals are very hygroscopic and corrosive and will burn and stain the skin. This is then precipitated out into a purple powder with a variety of compounds such as tin chloride, or potassium carbonate, washed several times and filtered with a glass filter in a Buchner funnel.

A solvent is prepared by sublimating Sal Ammoniac (NH₄Cl) until yellow, orange, or red crystals form. These crystals are collected and placed in a beaker that can be hermetically sealed. The beaker is filled with dehydrated alcohol (e.g., reacted with potassium carbonate crystals) and the entire contents are circulated for an extended period of time. The contents are then hermetically sealed. Once the extraction is complete from the circulation it is distilled three to seven times, continually pouring the liquid back into the retort together with the salt that didn't carry over. Once finished, the distillate is collected and saved in a cool place for use in extracting the oil from the precipitate.

To extract gold precipitate and solvent, a measured amount of prepared solvent is placed into a glass borosilicate flask to which is added a weighed quantity of the prepared gold precipitate (quantities are discussed hereinafter). The gold powder will slowly release its oil at a low to moderate temperature in the prepared solvent over several months. The reaction vessel is sealed and allowed to stand at room temperature for at least one month in the dark. A darkening of the solution will increase over time.

After the reaction time, the remaining oil is further refined by a variety of processes including severe temperature fluctuations, fractional distillation and extraction with a variety of alcohols, specifically ether, the prepared solvent, and all the alcohols can be reused by simply distilling and collecting. The final oil is then placed in 96% grain alcohol and used internally at a dose of 1-3 drops, or directly on the skin.

Relative proportions of prepared gold and solvent present a range of effective ratios which are time dependent. Less gold and more solvent increases the time required for the reactants to mature into a deep red amber product and precipitate the gold as metal. More gold and less solvent react more rapidly. In addition, the prepared solvent can be extended by dilution with chemical grade acetone which has been redistilled from glass at least once. The following examples illustrate successful mixtures for the production of “the oil of gold”.

In an example, 5 grams precipitated gold powder and 20 ml prepared solvent from sublimated sal ammoniac are sealed in a flask, allowed to circulate for at least 4 months, then filtered for use. Yields vary between 5 ml and 10 ml of viscous amber red liquid and 100% recovery of the gold. Dilution with ethanol produced a golden yellow tincture for use.

A unique substance may be formed by extraction of gold precipitate with a specially prepared solvent with sublimated sal ammoniac, eventually creating “the oil of gold”. As there is no standard in the industry for this preparation, the usual method of distribution is in the form of an alcoholic dilution or “tincture” standardized by depth of color against a known quantity of oil in ethanol as set by the end user. As little as 1 drop of oil per dram (4 ml) of ethanol has proved effective.

Although this is a product for research purposes and no clinical trials have been reported, anecdotal evidence from individuals indicate many possible medical benefits. These reports include relief from problems associated with diabetes, high blood pressure, heart attack, hepatitis C, acute myelytic leukemia, and cancer. As a preventative or dietary supplement, reports include mental clarity, and increased stamina.

An ozonated fluid or oil may be prepared using an ozone generator. By bubbling ozone gas through an ozone resistant container such as glass, the ozone gas is trapped in any oil to create a variety of oxygen combinations. In essence, what is taking place is a catalytic reaction that actually stores a form of ozone for an indefinite time period. The result yields ozonated oil which can be used topically and be ingested for beauty and healing purposes.

In a method of preparing the ozonated fluid, a glass container is half filled with a variety of plant, metal, or mineral based oils. A silicone or teflon tube is connected to an ozone resistant air stone, with the other end connected to the ozone output of the ozone generator. Additional silicone or teflon tubing is connected to an oxygen concentrator outlet (a supply of medical grade oxygen may be used) and the other end to an air intake of the ozone generator. Both the ozone generator and the oxygen concentrator are operated. An aerator stone is placed in the oil and visually checked for ozone bubbling out of the air stone. A distinctive odor of ozone should be observed.

Ozone may be bubbled through the oil until the oil turns into an expanding white foam or attains a jelly-like consistency. A minimum continuous run time of 3 to 12 weeks is preferred. The time required is dependent on the concentration of ozone used. After the foam settles and becomes a cream, the process is complete. Operation of the ozone generator and oxygen concentrator is discontinued, and the aeration stone is removed from the stainless vessel.

The oil, which may be a high quality plant oil such as olive oil, is poured into small glass jars that are safe for highly corrosive liquids. Lids are screwed onto the jars, which are then placed in refrigeration for storage. Ozonated olive oil, kept refrigerated, retained its effectiveness for over ten years, in tests conducted by German researchers. Other oils which may be used include tamanu oil, coconut oil, jojoba bean oil, sesame oil, sunflower oil, peanut oil, and egg and metal oils (prepared as described above).

It is recommended that any unused ozone be maintained under refrigeration.

These ozonated oils are desirable for their antibacterial properties as well as their healing properties. The oils can be added to any skincare product. Historically, the benefits the ozone oils are applied to insect bites, wrinkles, wounds, sores, skin infections, rash, and scars.

Oil of gold may be prepared through the catalytic effect of gold chloride on a specially prepared solvent mainly consisting of acetone. For various reasons, the resulting product has been labeled as “The Oil of Gold”. Pure gold is dissolved into aqua regia and the resulting solution is filtered through a 40 micron glass frit. The filtered liquid is slowly evaporated on a low heat until it crystallizes. The crystals are then recrystallized from a 1M solution of hydrochloric acid and then recrystallized from distilled water. The resulting deep golden orange crystals of Hydrogen tetrachloroaurate are then gently surface dried and stored in an airtight glass container kept cool and out of direct light. They are very hygroscopic and corrosive and will burn and stain the skin.

The solvent is prepared by the dry distillation of one of the selected metal acetates gradually up to a maximum temperature of 700 degrees Celsius. Special attention is paid to the cooling system used to condense the vapor produced in the distillation. The collected distillate is placed into a fractional distillation train and the fraction distilling over up to 56 degrees Celsius is collected. The resulting clear, volatile liquid is preserved in a sealed glass container stored in a cool place.

A measured amount of prepared solvent is placed into a glass flask to which is added a weighed quantity of the prepared gold crystals. The gold crystals will dissolve with a darkening of the solution which increases over time. The reaction vessel is sealed and allowed to stand at room temperature for at least one month in the dark.

After the reaction time, the solution will contain a precipitate of metallic gold. This may also appear in the form of metallic gold foil coating the glass vessel. The now dark amber red solution is filtered away from the gold precipitate and is sealed in a clean glass vessel for aging. This solution of “oil of gold” can be used in various products neat or diluted into ethanol which is 95% or stronger concentration. Dilution into strong alcohol should cause no further precipitation in all proportions. Additional aging improves the quality of the gold solution, similar to the aging and maturing of a fine wine.

In the above method, a preferred source is gold that has not been melted, thus placer mined nuggets or flakes are the best source. Pure gold which has been refined by fire can also be used. Gold of lesser karat value than 24 must be refined, with refinement through the use of antimony preferred. The solvent used in this process is prepared by the destructive distillation of a metal acetate salt. The preferred salt is lead acetate but the acetates of zinc and sodium may also be used.

Relative proportions of prepared gold and solvent present a range of effective ratios which are time dependent. Less gold and more solvent increases the time required for the reactants to mature into the deep red amber product and precipitate the gold as metal. More gold and less solvent react more rapidly. In addition, the prepared solvent can be extended by dilution with chemical grade acetone which has been redistilled from glass at least once. The following examples illustrate successful mixtures for the production of “The Oil of Gold”.

In a currently preferred example, 20 grams prepared gold is used with 10 ml freshly redistilled volatile from lead acetate. The mixture is sealed in flask and allowed to stand 2 months, then filtered for use. Yield is 8 ml of viscous amber red liquid and 100% recovery of the gold. The “oil” was capable of dilution with ethanol to several liters of useable tincture of a brilliant, golden yellow color and transparent. It will fluoresce under short wave UV light.

In another example, 5 grams prepared gold is mixed with 20 ml freshly redistilled volatile from sodium acetate, is sealed in a flask, allowed to stand for 4 months, then filtered for use. Yield was 18 ml viscous amber red liquid and 100% recovery of the gold. Dilution with ethanol produced a golden yellow tincture for use.

In a further example, 4 grams of prepared gold is mixed with 100 ml commercial acetone (glass distilled) and 2 ml freshly redistilled volatile from zinc acetate. The mixture is sealed in a flask, allowed to stand 4 months, then is filtered for use. Commercial acetone used alone requires several years to develop the same qualities as those prepared with the volatile derived from a metal acetate. The addition of a small amount of such a volatile (in this case from zinc) visibly accelerated the production of the deep amber red product.

As there is no standard in the industry for this preparation, the usual method of distribution is in the form of an alcoholic dilution or tincture standardized by depth of color against a known quantity of oil in ethanol as set by the end user. As little as 1 drop of oil per dram (4 ml) of ethanol has proved effective.

While the present invention has been described in connection with what is considered the most practical and preferred embodiment, it is to be understood that the present invention is not to be limited to the disclosed arrangements, but is intended to cover various arrangements which are included within the spirit and scope of the broadest possible interpretation of the appended claims so as to encompass all modifications and equivalent arrangements which are possible.

I claim:

1. A method of preparing a personal care substance in fluid form, comprising:
comminuting and calcining a gemstone;
forming a tincture from the comminuted and calcined gemstone by adding an alcohol solvent; and
removing the alcohol solvent by distillation, thereby leaving a liquid thicker than the solvent.

2. The method of claim 1, further comprising adding alcohol of at least 96% strength to form a tincture.

3. The method of claim 1, further comprising:
extracting ashes from the thicker liquid using filtered rainwater;
filtering the mixture and allowing crystals to form;
blending the filtered mixture and crystals with tincture;
removing alcohol by distillation in a plurality of iterations;
maintaining the mixture at 110 degrees F. for several months;
filtering the liquid; and
removing solids.

4. A method of preparing a mineral fulvate complex, comprising:
adding a solution of sodium hydroxide and water to ocean water until a pH of 10.5 is reached;
allowing the mixture to settle;
removing supernatant;
washing precipitate; and
adding fulvic or humic acid to lower the pH.

5. The method of claim 4, wherein adding fulvic or humic acid comprises adding the fulvic or humic acid in individual drops.

6. A method of preparing an oily product through the extraction of a pure gold precipitate, comprising:
sublimating sal ammoniac (NH₄Cl) containing gold dissolved into aqua regia several times until a yellow or orange crystal forms;
filtering the resulting solution;
evaporating the filtered liquid until it crystallizes; and
removing crystals of hydrogen tetrachloroaurate to leave a remaining oil.

7. The method of claim 6, additionally comprising further refining the remaining oil by at least one of severe temperature fluctuations, fractional distillation and extraction with a variety of alcohols.

8. (canceled)

9. (canceled)
